(19) United States
(12) Patent Application Publication
(10) Pub. No.: US 2019/0254599 A1
(43) Pub. Date: Aug. 22, 2019

(54) SYSTEMS AND METHODS FOR NON-INVASIVE MONITORING OF ANIMALS

(71) Applicant: Sentier HC LLC, Milwaukee, WI (US)

(72) Inventors: Robert L. Young, Waukesha, WI (US); William Merrick, Milwaukee, WI (US); Keith Jasinski, Milwaukee, WI (US); Kyle McFarlane, Madison, WI (US)

(21) Appl. No.: 16/266,748

(22) Filed: Feb. 4, 2019

Related U.S. Application Data
(60) Provisional application No. 62/625,664, filed on Feb. 2, 2018.

(51) Int. Cl. A61B 5/00; A01K 29/00; A61B 5/11; A61B 5/145

(52) U.S. Cl. CPC A61B 5/6805; A01K 29/005; A61B 5/1118; A61B 5/1455; A61B 5/0006; A61B 5/4519; A61B 2562/0219; A61B 5/14532

(57) ABSTRACT

Systems and methods for non-invasive monitoring of animals are provided. In some aspects, a method for monitoring an animal subject is provided. The method includes receiving an indication identifying an animal subject to be monitored, and selecting a plurality of monitoring parameters in accordance with the indication. The method also includes monitoring the animal subject for a predetermined period of time using the plurality of monitoring parameters, and generating a report.

### CROSS-REFERENCE TO RELATED APPLICATIONS

This application claims the benefit under 35 U.S.C. § 119(e) of co-pending U.S. Provisional Patent Application Ser. No. 62/625,664, filed Feb. 2, 2018, which is hereby incorporated by reference.

### GOVERNMENT RIGHTS

N/A

### BACKGROUND

The present disclosure relates generally to systems and methods for patient monitoring. More particularly, the present disclosure relates to systems and methods for non-invasive monitoring of animals.

Technologies for monitoring the vital signals of a patient that are currently available have many limitations. For instance, many monitoring systems are bulky, costly, and therefore used mostly in clinical settings. By contrast, portable monitoring devices, such as activity and health trackers, are more practical and cost-effective, but often provide only specific vital signals, such as heart rate, temperature or activity. Furthermore, such portable monitoring devices are not suitable for animal patients. This is because animal patients introduce a number of technical challenges for health monitoring, including the ability to establish and maintain reliable measurements, particularly when the animal is conscious or active. For example, animal hair, skin thickness, and skin pigmentation present a number of difficulties for obtaining measurements using current health monitors. In addition, the stresses that the clinical setting causes for the animal patients can yield measurements that do not reflect normal values for that animal.

Animal patient monitoring outside a clinical setting can be advantageous in reducing veterinary costs, and allow longer-term health monitoring without the stresses of hospital visits. Therefore, there is a need for animal-friendly monitoring systems that are cost effective, easy to use and can simultaneously and non-invasively monitor multiple vital signals of patients, especially non-human patients.

### SUMMARY

The present disclosure overcomes the drawbacks of previous technologies by introducing systems and methods for monitoring animals non-invasively.

In one aspect of the present disclosure, a system for monitoring an animal subject is provided. The system includes a sensor assembly comprising a plurality of sensors configured to detect at least physiological signals from an animal subject, and a signal detector configured to generate signal data corresponding to the physiological signals detected by the plurality of sensors. The system also includes a monitor unit comprising a controller configured to receive the signal data generated by the signal detector. The controller is also configured to analyze the signal data to determine an indication identifying the animal subject, and select a plurality of monitoring parameters in accordance with the indication. The controller is further configured to control the monitor unit to monitor the animal subject for a predetermined period of time using the plurality of monitoring parameters, and generate a report.

In another aspect of the present disclosure, a method for monitoring an animal subject is provided. The method includes receiving an indication identifying an animal subject to be monitored, and selecting a plurality of monitoring parameters in accordance with the indication. The method also includes monitoring the animal subject for a predetermined period of time using the plurality of monitoring parameters, and generating a report.

In yet another aspect of the present disclosure, a system for monitoring an animal subject is provided. The system includes a sensor assembly comprising a plurality of sensors configured for detecting at least physiological signals from a skin of an animal subject, and a signal detector configured to generate signal data corresponding to the physiological signals detected by the plurality of sensors. The system also includes a controller configured to control the signal detector to monitor the animal subject.

The foregoing and other advantages of the present disclosure will appear from the following description.

### BRIEF DESCRIPTION OF THE DRAWINGS

FIG. 1 a schematic diagram of an example monitoring system, in accordance with aspects of the present disclosure.

FIG. 2A is one embodiment of the monitoring system of FIG. 1.

FIG. 2B is another embodiment of the monitoring system of FIG. 1.

FIG. 3A is an illustration showing example of a harness for use with a monitoring system, in accordance with aspects of the present disclosure.

FIG. 3B is an illustration showing another example of a harness for use with a monitoring system, in accordance with aspects of the present disclosure.

FIG. 4A is a diagram showing a first portion of the harness shown in FIG. 3B.

FIG. 4B is a diagram showing a second portion of the harness shown in FIG. 3B.

FIG. 5A is a side view of an electrode, in accordance with aspects of the present disclosure.

FIG. 5B is a sectional view of the electrode shown in FIG. 5A.

FIG. 5C is an exploded perspective view of the electrode shown in FIG. 5A.

FIG. 6A is a schematic diagram showing one embodiment of a sensor having an implantable unit, in accordance with aspects of the present disclosure.

FIG. 6B is a schematic diagram showing another embodiment of a sensor having an implantable unit, in accordance with aspects of the present disclosure.

FIGS. 7A-7D are illustrations showing non-limiting example outputs displayed by the monitoring system of FIG. 1.

FIGS. 8A and 8B are illustration showing non-limiting examples of the monitoring system of FIG. 1.

FIG. 9 is an illustration showing an example monitoring network, in accordance with aspects of the present disclosure.

FIG. 10 is a flowchart setting forth steps of a process, in accordance with aspects of the present disclosure.

### DETAILED DESCRIPTION

The description below and the accompanying figures provide a general understanding of the environment for system and methods disclosed herein as well as the details for the system and methods. In the drawings, like reference numerals are used throughout to designate like elements.

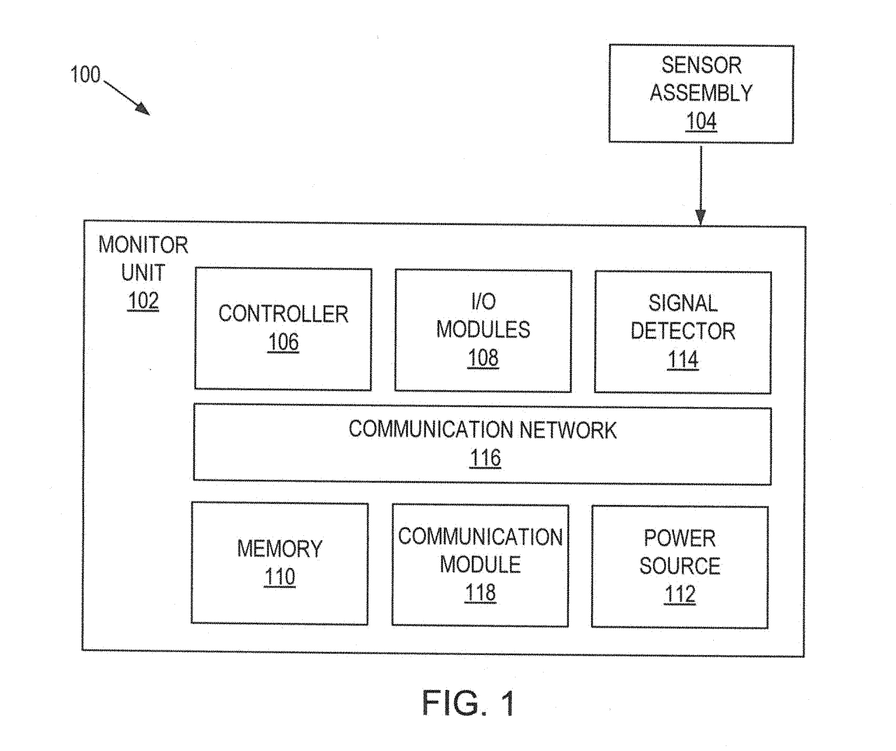

Turning to FIG. 1, a monitoring system 100, in accordance with aspects of the present disclosure, is shown. In general, the monitoring system 100 may include a monitor unit 102 and a sensor assembly 104. The monitor unit 102 may include a controller 106, a number of input/output (“I/O”) modules 108, a memory 110, a power source 112, and a signal detector 114. As shown, the monitor unit 102 may also include a communication network 116 configured to facilitate the transfer of data, signals and other information between the various elements of the monitoring system 100. The monitor unit 102 may also include a communication module 118 allowing data and information exchange with any external device, device hub, system, database, server, internet, cloud, and so on. Above-mentioned elements in the monitor unit 102 may be contained in a single or multiple housing units.

The monitoring system 100 may operate autonomously or semi-autonomously, using information, data or instructions provided directly by a user using the I/O modules 108, or communicated via the communication module 118. In some embodiments, the monitoring system 100 may be a stand-alone, wearable, or portable device. In addition, the monitoring system 100 may be preferably designed, shaped and dimensioned to monitor animal subjects non-invasively, with minimal interference.

As will be described, the sensor assembly 104 may include a variety of sensors, sensor configurations, and substrates for detecting physiological signals, vital signs, and other signals indicative of a subject's health or condition. Non-limiting examples of sensors include electrocardiogram (“ECG”) sensors, blood pressure sensors, optical sensors, oxygenation sensors, oxygen saturation sensors, respiration sensors, temperature sensors, glucose sensors, muscle activity sensors, and so on. As such, sensors in the sensor assembly 104 may include various electrodes, optical sources and detectors, pressure components, force components, chemical detectors, and others. In some implementations, the sensor assembly 104 may include sensors for determining the subject's activity, such as motion, impact and duration of activity, and so on. Such sensors may therefore include motion sensors (e.g. accelerometers), location sensors (e.g. GPS sensors), and others.

In some preferred embodiments, the sensor assembly 104, and sensors therein, may be configured for detecting and monitoring animal subjects, such as companion animals, domestic animals, lab animals, wild animals, and so on. As such, the sensors may be configured for reliably obtaining measurements by overcoming or adapting to difficulties associated animal hair, skin thickness and skin pigmentation, and so on. In one example, as will be described, “wet” electrodes may be used to detect electrical signals from an animal subject having hair or fur. In another example, optical sensors operating in frequency ranges or wavelengths that correspond to an animal subject's skin pigmentation may be used. In yet another example, sensors may be configured to measure signals from animal subjects having various skin thicknesses. For instance, animals with thick skin may require specialized sensors for enhancing, and/or processing electrical and/or optical signals.

In general, the controller 106 may be configured to carry out various steps to operate the monitoring system 100, including controlling elements of the monitoring system 100, data acquisition, data transfer, data storage, data retrieval, data processing, reporting, power management, and so on. In particular, the controller 106 may be configured to control the signal detector 114 to acquire signals detected by sensors on the sensor signal assembly 104. For instance, the controller 106 may control the intermittent or continuous acquisition of ECG, optical, and other signals. The controller 106 may also be configured to control the assembly of signal data, for instance as waveforms, and process the signal data, for instance to generate various vital signs and signatures indicative of the subject's health. For example, the controller 106 may compute specific indices, such as a pain index. In some implementations, the controller 106 may analyze various vital signs and signatures and determine a condition of the subject. In so doing, the controller 106 may take into consideration various information, including specific information associated with the subject being monitored (e.g. age, animal type, prior medical history, and so on). For example, the controller 106 may detect seizures, or determine non-compliance with doctor instructions (e.g. cage rest) The controller 106 may then generate and provide a report accordingly. The controller 106 may also be configured to control the monitoring of a subject for a predetermined period of time. For example, an animal subject may be monitored intermittently or continuously for several minutes, hours, days or weeks, as needed.

In some implementations, the controller 106 may include one or more processing units, such as microcontrollers, microprocessors, and others, that may be configured to execute non-transitory programming, or instructions hardwired therein. Such processing units would therefore be application-specific. Alternatively, or additionally, the controller 106 may include one or more general-purpose processors configured to access and execute instructions stored in non-transitory, as well as transitory, computer readable media in the memory 110. By way of example, general-purpose processors may include central processing units (“CPUs”), graphical processing units (“GPUs”), and the like. In some implementations, the controller 106 may optionally execute steps based on operational instructions or selections provided by a user.

In some aspects, the controller 106 may be configured to execute steps, in accordance with methods of the present disclosure. In some aspects, the controller 106 may be configured to analyze signal data acquired using the signal detector 114, as well as other information, and adapt or modify operation of the monitoring system 100 based on the analysis. For instance, based on detected vital signs or physiological signals, the controller 106 may identify the animal subject to be monitored. Alternatively, or additionally, and/or an indication identifying the animal subject to be monitored may be received from a user. The controller 106 may then select a plurality of monitoring parameters or conditions for monitoring based on the identified subject. Specifically, the controller 106 may select particular sensor configurations and acquisition settings, and control the signal detector 114 and/or sensor assembly 104 accordingly. For example, the controller 106 may select specific combinations of sensors and signals to be acquired, and adapt sampling of those signals. In addition, the controller 106 may also select various display settings in accordance with the animal subject.

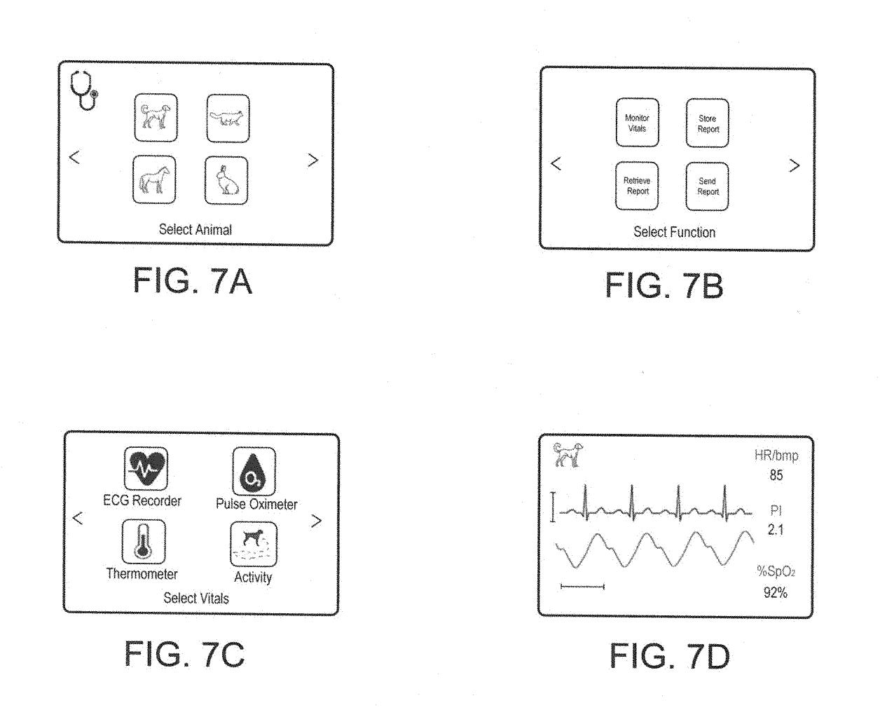

In some implementations, the controller 106 may use the I/O modules 108 to prompt a user to provide information with regard to an animal subject to be monitored, as well as actions to be taken. By way of example, FIGS. 7A-7C illustrate screen shots prompting a user to select an animal subject (e.g. dog, cat, horse, rabbit, and so on), to select a function to be carried out (e.g. monitor vitals, store report, retrieve report, send report, and so on), and select vitals to be monitored (e.g. ECG, pulse oximeter, thermometer, activity, and so on). It may readily be understood that these are mere examples, and in no way limit the present disclosure.

The controller 106 may also process/output the acquired signals based on the identified animal subject. For example, the controller 106 may select specific amplification, scaling, and filtering for the acquired signals. The controller 106 may also compute different waveforms, parameters or indices using the signals, as well as report or display different information, data or signals, depending upon the identified animal subject. For example, the controller 106 may control a display of acquired signals using amplitude and temporal scales specific to the animal subject identified. A non-limiting example is shown in FIG. 7D. It may readily be understood that the example of FIG. 7D will in no way limit the present disclosure.

The I/O modules 108 of the monitoring system 100 may be configured to receive a wide variety of data, information, as well as selections, and operational instructions from a user. To this end, the I/O modules 108 may include various elements for receiving input, including buttons, switches, toggles, knobs, touch screens, or other touch-responsive elements, as well as ports, connectors, and receptacles for flash-memory, USB sticks, cables, and so on. The I/O modules 108 may also be configured to provide a report by way of various output elements, including screens, displays, LEDs, LCDs, speakers and so on.

The memory 110 may include various memory elements where a number of types of data (e.g., internal data, external data instructions, software codes, status data, diagnostic data, etc.) may be stored. In addition, as shown in FIG. 1, the memory 114 may also include non-transitory computer-readable media, which may include executable instructions or programs for carrying out methods in accordance with present disclosure. In some implementations, the memory 110 may store predetermined configurations for operating the monitoring system 100. For instance, the memory 110 may include various sensor and data acquisition configurations tabulated based on various animal subjects. Example elements in the memory 110 may include random access memory (“RAM”), dynamic random-access memory (“DRAM”), electrically erasable programmable read-only memory (“EEPROM”), flash memory, and the like.

The power source 112 is configured to power various elements and circuitry in the monitor unit 102, as well as the sensor assembly 104. For example, the power source 112 may include a rechargeable or replaceable battery.

The signal detector 114 may include a wide variety of elements, circuitry and hardware. In particular, the signal detector 114 may be configured to receive or sample signals from sensors on the sensor assembly 104. Signals received by the signal detector 114 may be analog as well as digital signals. For example, the signals may be in the form of voltage signals, current signals, optical signals, and so on. The signal detector 114 may also include capabilities for processing the received or sampled signals. Specifically, the signal detector 114 may include capabilities for amplifying, scaling, filtering, and digitizing the received signals.

The communication network 116 may include a variety of communication capabilities and circuitry, including various wiring, components and hardware for electronic, radiofrequency (“RF”), optical and other communication methods. By way of example, the communication network 116 may include parallel buses, serial buses, and combinations thereof. Example serial buses may include serial peripheral interface (SPI), I2C, DC-BUS, UNI/O, 1-Wire, and others. Example parallel buses may include ISA, ATA, SCSI, PIC, IEEE and others.

The communication module 118 may be configured to facilitate communications between the monitoring system 100 and various external devices. To this end, the communication module 118 may include any hardware, software, and firmware capable of achieving wired or wireless communication. In some implementations, the communication module 118 may be configured perform wireless communication using RF, Bluetooth or other wireless communication protocols.

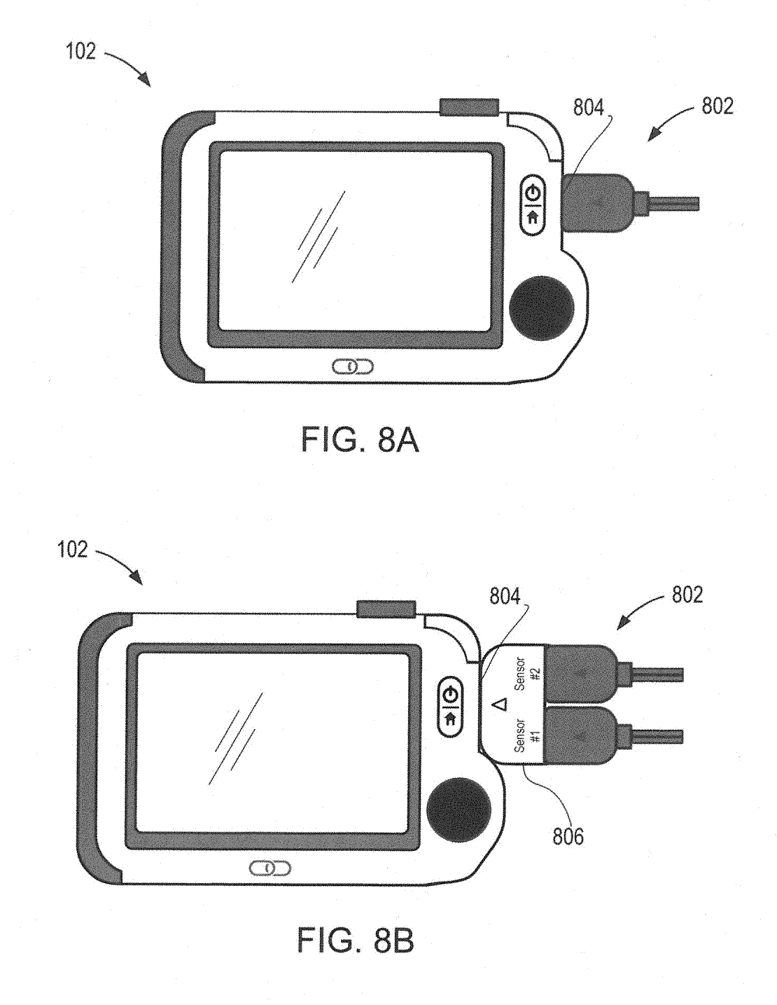

By way of example, FIGS. 8A and 8B illustrate a non-limiting example of a monitoring unit 102, as described above. In particular. FIG. 8A shows a monitoring unit 102 configured to receive one input connection 802 from a sensor assembly via an input receptacle 804. In one example, the input connection 802 may provide ECG signals. On the other hand, FIG. 8B shows the monitoring unit 102 configured to receive more than one input connection 802 via the input receptacle 804. To do so, an adapter 806 may be optionally used, as shown. It may be appreciated however, the input receptacle 804 may be configured to receive multiple input connections 804 without need for an adapter 806.

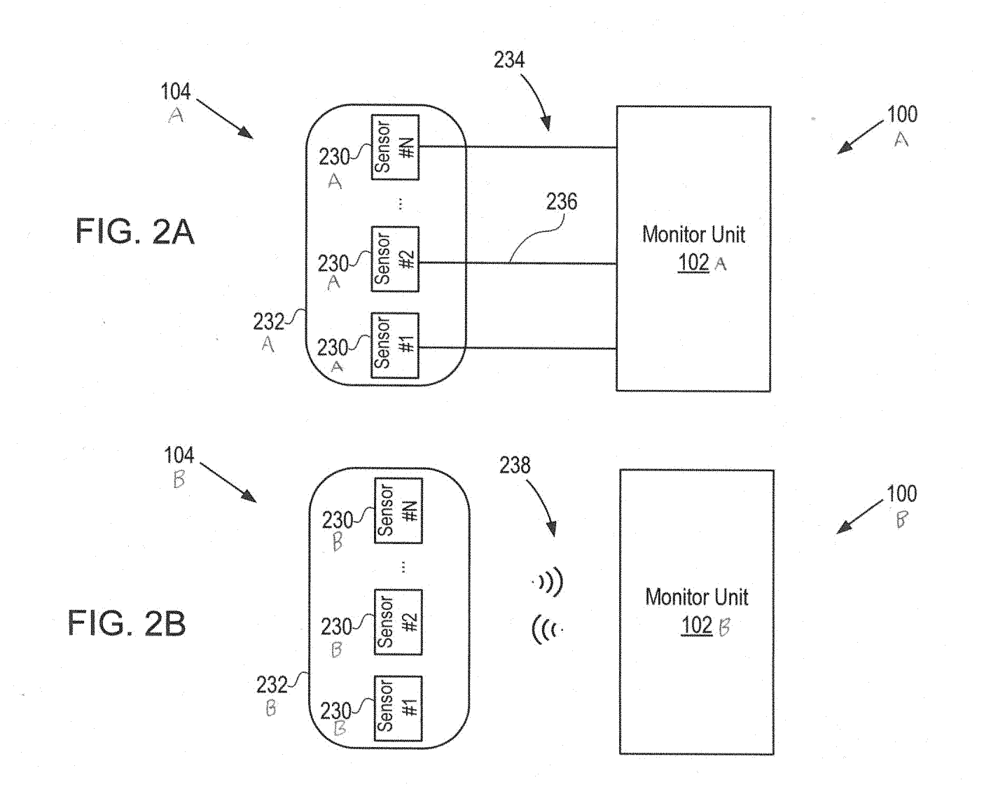

In some embodiments, the sensor assembly 104 of FIG. 1 may include a number of sensors 230 arranged on a substrate 232, as shown in FIGS. 2A and 2B. Specifically, the sensors 230 may be coupled to the substrate 232, and arranged in any manner and configuration, depending upon the functionality and/or signals acquired by the sensors 230. In one embodiment, the sensors 230 include two or more ECG sensors (e.g. ECG electrodes, ECG electrode assemblies, and so on) arranged on the substrate 232 at locations advantageous for sensing heart activity of the subject to be monitored. In another embodiment, the sensors 230 include at least one respiratory sensor arranged on the substrate 232 at location(s) advantageous for sensing respiration. In yet another embodiment, the sensors 230 include at least one blood pressure sensor, arranged on the substrate 232 at location(s) advantageous for sensing blood pressure. As may be appreciated, a number of embodiments including various sensor combinations may be possible.

As shown in FIG. 2A, the sensors 230A in the sensor assembly 104A may communicate with the monitor unit 102A using a wired communication link 234 that may include a number of sensor connections 236. For example, the sensor connections 236 may include fixed or removable optical or electrical conduits or wires. Alternatively, or additionally, the sensors 230B may communicate with the monitor unit 102B using a wireless communication link 238, as shown in FIG. 2B. To this end, the sensors 230B may include capabilities for telemetry or wireless communication.

As shown in FIGS. 2A and 2B, the substrate 232 of the sensor assembly 104 may be configured to engage the sensors 230 to the subject, and more specifically an animal subject. To this end, the substrate 232 may be configured to conform, at least in part, to the subject's anatomy, or a portion thereof. In addition, the substrate 232 may have various shapes and dimensions, and be manufactured using various flexible, rigid, or semi-rigid materials. For example, the substrate may be manufactured using polyoxymethylene (“POM”) materials (e.g. acetal, polyacetal, polyformaldehyde, and so on), rubber-like materials (e.g. TangoGray™, TangoBlack™, TangoPlus™, TangoBlackPlus™, and so on), photopolymer materials, plastic materials and so on. In some implementations, the substrate 232 may be manufactured using a material that is non-conductive. This would allow electrical conductors (e.g. ECG electrodes) coupled to the substrate 232 to be electrically isolated from one another. The substrate 232 may also include a variety of other elements, or components, including circuitry (e.g. rigid or flexible circuits), and so on.

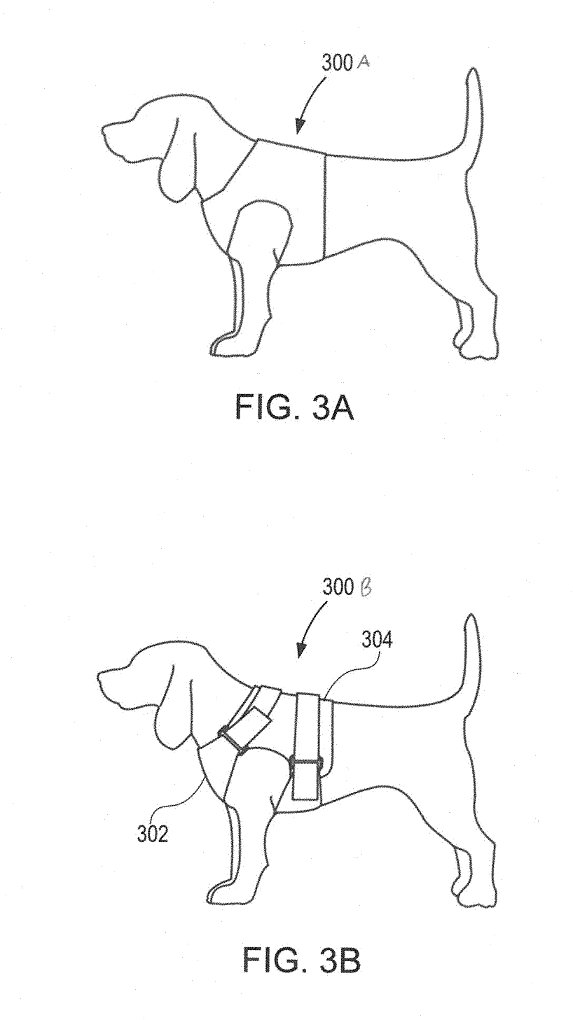

The sensor assembly 104 described with reference to FIGS. 2A and 2B may be configured to attach directly to the animal subject. Alternatively, or additionally, the sensor assembly 104 may be part of; attached to, or incorporated into a wearable item or product that may be coupled to the animal subject. For instance, the sensor assembly 104 may be attached to, or part of, a harness 300A or 3008, as shown in the examples of FIGS. 3A and 3B. The sensor assembly 104 may also be attached to, part of, or incorporated into a collar, a belt, a strap a vest, or other wearable item. Referring particularly to the harness 300A shown in FIG. 3A, the harness 300A may be formed using a single piece, which may be slipped over the head of the animal subject and secured in place using various fasteners, including belts, clips, snaps, straps, buttons, Velcro, and others. Alternatively, as shown in FIG. 3B, the harness 300B may include a first portion 302 and a second portion 304, which may also be coupled together using various fasteners. The first portion 302 and second portion 304 may be configured to fit an animal subject's anatomy, as shown.

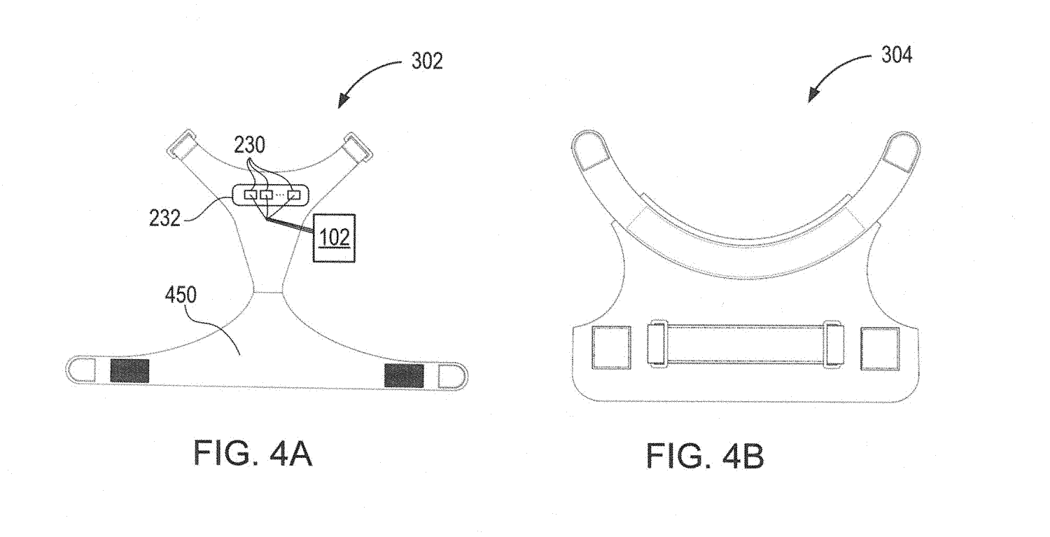

The interior part 450 of the first portion 302 of the harness 300′ in FIG. 3B is shown in FIG. 4A, The interior surface 450 represents the side of the first portion 302 that makes contact with the animal subject. As shown in FIG. 4A, the substrate 232 may be attached to the interior surface 450, allowing sensors 230 coupled therein to make contact with the animal subject when the first portion 302 is fastened to the second portion 304 (FIG. 4B). The sensor assembly 104 may be permanently or removably attached to the interior surface 450, for example, using Velcro. Wires or cables from sensors 230 in the sensor assembly 104 may extend parallel to interior surface 450 and connect to a monitor unit 102, as shown in FIG. 4A. Alternatively, wires or cables from the sensors 230 may penetrate the interior surface 450.

As described, the sensor assembly 104 of FIGS. 2A and 2B may be coupled to an animal subject to acquire physiological signals, vital signals, and other information. However, many animal subjects have fur that can impede the ability to reliably detect desired signals, such as electrical signals required for ECG measurements. As such, it is often the practice that fur needs to be removed from areas of measurement on the animal, which can be cumbersome or undesirable. To avoid this issue, a solution is introduced herein based on a “wet” electrode concept, as described below.

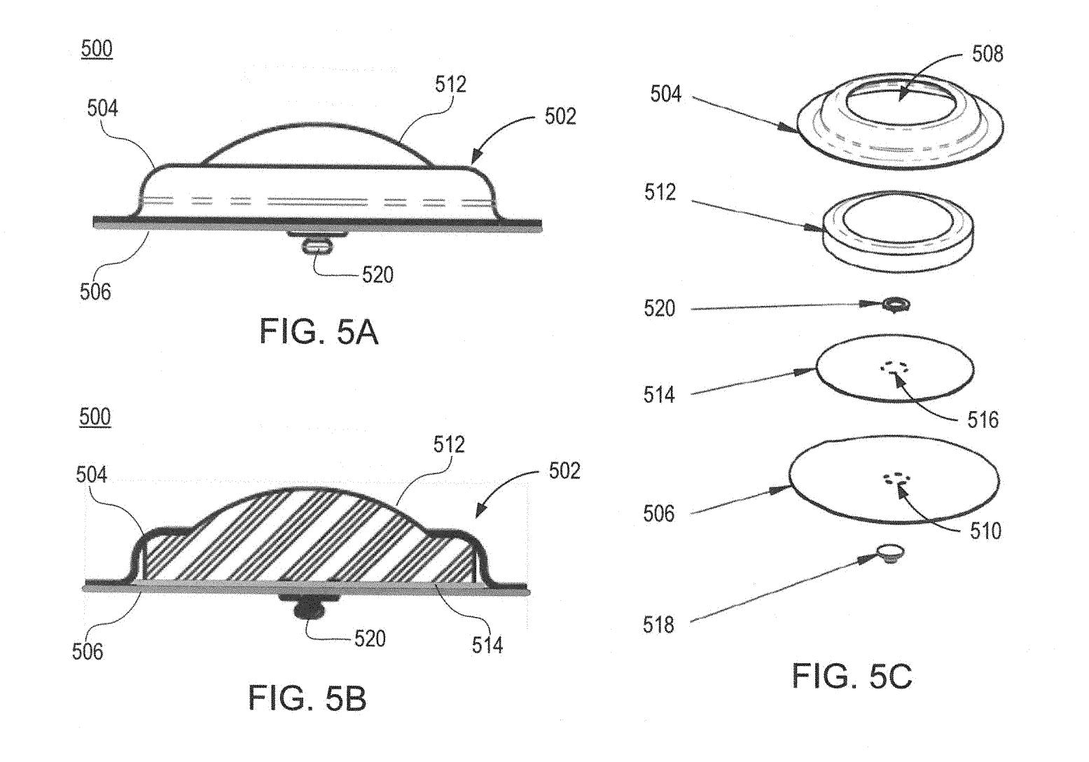

Referring specifically to FIGS. 5A-C, an example electrode 500, in accordance with aspects of the present disclosure, is shown. The electrode 500 may be attached to a substrate 232, or incorporated into a sensor assembly 104, as described with reference to FIGS. 2A and 2B. Alternatively, the electrode 800 may be attached directly to an interior of a harness 300A or 300B, as described with reference to FIGS. 3A and 3B.

As shown, the electrode 500 includes a housing 502 having a front wall 504 and a back wall 506. The front wall 504 includes an opening 508 that is substantially in the center of the front wall 504. The back wall 506 includes a first through hole 510 that is substantially the center of the back wall 506. The front wall 504 and the back wall 506 enclose a main body 512 within the housing 502, with a portion of the main body 512 protruding through the opening 508 in the front wall 504. In one specific embodiment, the back wall 504 may also include a means of attachment, such as an adhesive layer or a Velcro material, positioned on an exterior portion of the back wall 504. This would allow attachment to a substrate or harness, as described.

The housing 502 may be shaped and dimensioned to provide mechanical support to the main body 512. In addition, the housing 502 may be configured to substantially retain the main body 512 in place when pressure is placed thereupon as a result of bringing the electrode 500 into contact with the subject. The housing 502 may be manufactured using various materials, including polymer materials, thermoformed materials, plastics, Teflon, and so on. In some implementations, the housing 502 may be non-conductive. The front wall 504 and back wall 506 may be manufactured using similar or different materials. In assembling the electrode 500, the outer edge of the front wall 502 may be attached to the outer edge of the back wall 506 using various methods knowing in the art, including applying adhesives or glue, heat sinking, heat welding, or solvent welding.

The electrode 500 also includes a conducting layer 514 in contact with the back wall 506. The conducting layer 514 includes a second through hole 516 that is substantially the center of the conducting layer 514. By way of example, the conducting layer 514 may include a metallic substrate or mesh (e.g. stainless steel mesh). Electrical contact may be established between the conducting layer 514 and a conducting pin 518 by fastening the conducting pin 518 to a fastener 520. The conducting pin 518 may be used to connect to a monitoring unit, as described with reference to FIG. 1. In some implementations, the fastener 520 may also be electrically conducting. The conducting pin 518 and fastener 520 may form a snap assembly.

The main body 512 of the electrode 500 may be configured to absorb conductive liquid (e.g. water, gel, or other liquid). As appreciated, the conductive liquid would provide an electrical pathway between the conducting pin 518 and the subject. The main body 512 may be manufactured using a stretchable material, a porous material, a polymer, or a combination thereof. For example, the main body 512 may include a sponge or a similar material. This would allow electrical contact with an animal subject's skin without need for removing the animal's fur.

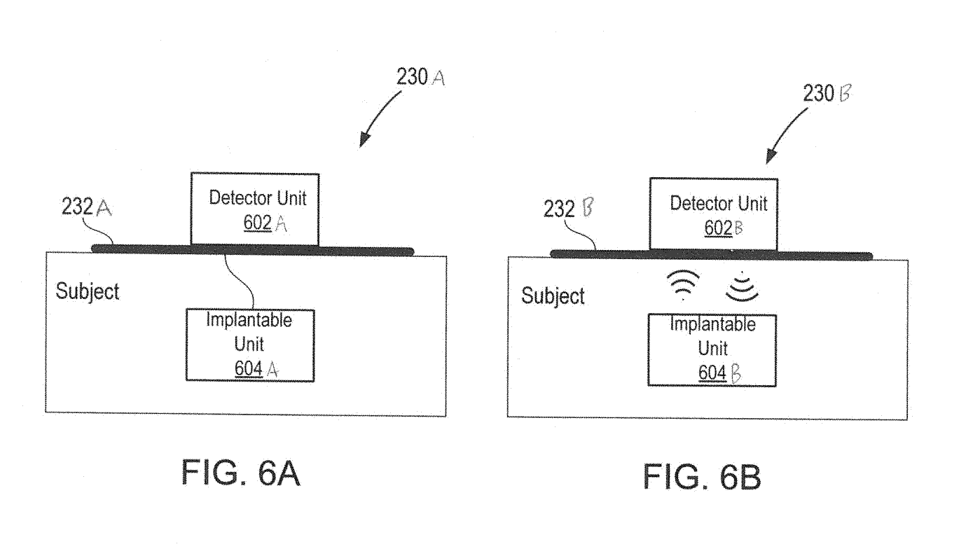

FIGS. 6A and 6B show other embodiments of the monitoring system 100. In particular, the figures illustrate an example sensor 230A or 230B that may be used in a sensor assembly 104, as described with reference to FIGS. 2A and 2B. In general, the sensor 230 may include a detector unit 602 and an implantable unit 604. The implantable unit 604A may be configured to detect various signals from the subject, and communicate detected signals using a wired connection, as described. Alternatively, the implantable unit 604B may be configured to communicate detected signals using a wireless connection, as shown. To this end, the detector unit 602B and implantable unit 604B may be configured with various hardware, including Bluetooth, or other wireless protocol.

The detector unit 602A or 602B may be coupled to a substrate 232A or 232B, respectively, which could be attached to the subject using a harness, as well as other wearable items or products, as described. Signals received by the detector unit 602A or 602B may be communicated to a monitoring unit 102, as described with reference to FIG. 1. The detector unit 602A or 602B may advantageously amplify, filter, or otherwise pre-process the received signals detected by the implantable unit 604A or 604B. In implementations, the detector unit 602A or 602B may be a device similar to the monitoring unit 102 of FIG. 1.

Systems, as described in the present disclosure, provide a number of advantages for monitoring animal patients non-invasively. For instance, a number of systems may be used in a network to provide a veterinary with real-time information for a number of animal patients.

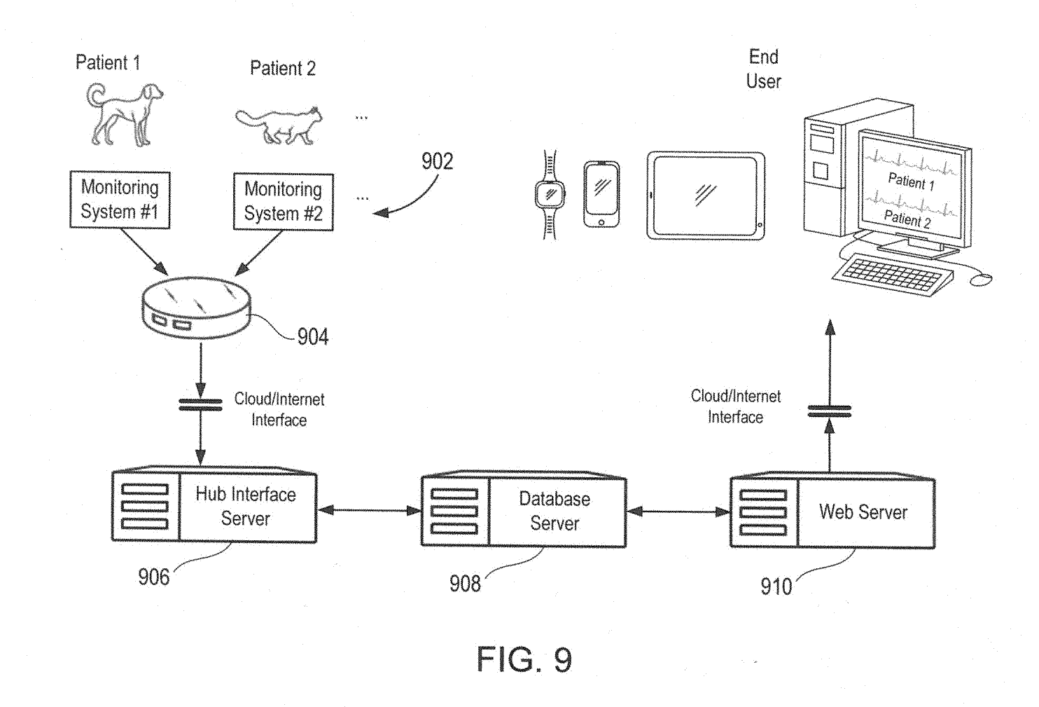

By way of example, FIG. 9 illustrates a monitoring network, in accordance with aspects of the present disclosure. The monitoring network may include a number of monitoring systems 902, which may relay data, signals and information to a hub 904. Monitoring systems 902 may be located close or far from one another. For example, a number of animals from a single household may be monitored. The hub 904 may include one or more devices capable providing access to the internet, including personal devices (e.g. smartphones, laptops, tablets and so on), routers, and so forth. In some implementations, the hub 904 can provide a bridge between the various monitoring systems 902 (e.g. BTLE) and the internet (e.g. wired LAN or wireless LAN). In some applications, it is envisioned that animals from more than one location or household may be monitored. As such, more than one hub 904 may be utilized.

The hub 904 shown in FIG. 9 may then upload data, signals and information received to a hub interface server 906 by way of a first internet/cloud interface. The hub interface server 906 may be a dedicated server configured to collect data and facilitate communicating and serving various devices/hubs. In some aspects, the hub interface server 906 can replicate and scale as the number of hubs increases. The data, signals and information may then be relayed to a database server 908. The database server 908 can also scale and replicate as the size of the monitoring network changes. In particular, the database server 908 may collect and store the data received through the hub interface server 906. In some aspects, the database server 908 may also send data, signals and information back to the hub interface server 906, the hub 904, as well as the monitoring systems 902. In this manner, configurations and settings of the monitoring systems 902 may be controlled or changed.

The database server 908 also relays data, signals and information to a dashboard web server 910, as shown. The web server 910 may then relay the data, signals and information to an end user (e.g. a veterinarian, pet owner, and so on) via a second cloud/internet interface. In this manner, an end user may receive the data, signals and information from a number of patients on a personal device, including a smart watch, smartphone, tablet, laptop, computer and so forth, and be able to take action accordingly. In some implementations, the web server 910 may be configured to provide a customized presentation or output of the data, signals and information to the end user. For example, the presentation may be in the form of a website, a dashboard, and so on, and include various features and information, including alerts or alarms indicating conditions for the monitored patients. The web server 910 may also be scaled with demand.

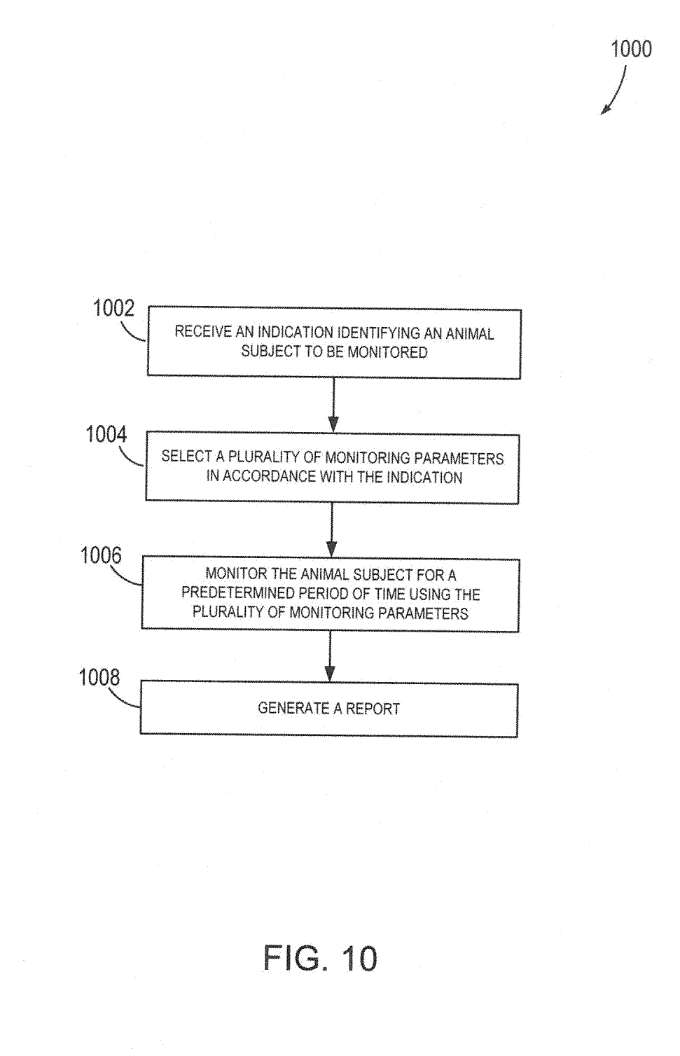

Turning now to FIG. 10, the steps of a process 1000, in accordance with aspects of the present disclosure, are shown. In some implementations, the process 1000 may be used to monitor an animal subject. In another implementations, the process 1000 may be used to generate a report indicating a condition of the subject. The process 200 may be carried using a monitoring system 100, as described with reference to FIG. 1, or another suitable system, device or apparatus. Steps of the process 1000 may be implemented as a program, firmware, or executable instructions hardwired or stored in non-transitory computer readable media.

The process 1000 may begin at process block 1002 with receiving an indication identifying an animal subject to be monitored. As described, the indication may be based on user input, and/or based on a determination made by a controller or processor by analyzing various acquired signals.

Then, at process block 1004, a plurality of monitoring parameters may be selected in accordance with the indication. The plurality of monitoring parameters may correspond to particular sensor configurations and/or acquisition settings. In particular, specific combinations of sensors and signals to be acquired may be selected at process block 1004. For example, a combination of ECG sensors, blood pressure sensors, optical sensors, oxygenation sensors, oxygen saturation sensors, respiration sensors, temperature sensors, glucose sensors, muscle activity sensors, motion sensors, location sensors, may be selected. In addition, sampling rates, filtering, amplification, scaling, display settings, other parameters may also be selected at process block 1004.

The selected plurality of monitoring parameters may then be used to monitor the animal subject for a predetermined period of time, as indicated by process block 1006. For example, an animal subject may be monitored intermittently or continuously for several minutes, hours, days or weeks, as needed. A report may then be generated as indicated by process block 1008.

The report may be in any form and includes any information. The report may be provided in substantially real time, for example, using a display, or stored in a memory to be retrieved at a later time. The report may also be uploaded to a database in the cloud, or transmitted over the internet to a device of an end user (e.g. a veterinarian). In some aspects, the report may be in the form of graphs, waveforms, time traces, indices, and so forth, corresponding to monitored physiological signals, vitals, activity, and so on.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. Various features and advantages of the invention are set forth in the following claims.

1. A system for monitoring an animal subject, the system comprising:
a. a sensor assembly comprising a plurality of sensors configured to detect at least physiological signals from an animal subject;
b. a signal detector configured to generate signal data corresponding to the physiological signals detected by the plurality of sensors; and
c. a monitor unit comprising a controller configured to:
i. receive the signal data generated by the signal detector;
ii. analyze the signal data to determine an indication identifying the animal subject;
iii. select a plurality of monitoring parameters in accordance with the indication;
iv. control the monitor unit to monitor the animal subject for a predetermined period of time using the plurality of monitoring parameters; and
v. generate a report.

2. The system of claim 1, wherein the plurality of sensors comprises a combination of electrocardiogram (“ECG”) sensors, blood pressure sensors, optical sensors, oxygenation sensors, oxygen saturation sensors, respiration sensors, temperature sensors, glucose sensors, and muscle activity sensors.

3. The system of claim 1, wherein the sensor assembly further comprises at least a plurality of motion sensors, and location sensors.

4. The system of claim 1, wherein the sensor assembly further comprises at least one electrode comprising a main body configured to absorb a conductive liquid providing an electrical pathway between a conducting pin on the at least one electrode and the animal subject.

5. The system of claim 4, wherein the main body is manufactured using a stretchable material, a porous material, a polymer, or a combination thereof.

6. The system of claim 1, wherein the sensor assembly further comprises a substrate, and wherein one or more of the plurality of sensors are attached to the substrate.

7. The system of claim 1, wherein one or more of the plurality of sensors comprises an implantable unit.

8. The system of claim 1, wherein the system further comprises at least one input module configured to receive input from a user.

9. The system of claim 8, wherein the controller is further configured to determine the indication based on the input from the user.

10. The system of claim 1, wherein the report indicates a condition of the animal subject.

11. A method for monitoring an animal subject, the method comprising:
a) receiving an indication identifying an animal subject to be monitored;
b) selecting a plurality of monitoring parameters in accordance with the indication;
c) monitoring the animal subject for a predetermined period of time using the plurality of monitoring parameters; and
d) generating a report.

12. The method of claim 11, wherein the method further comprises monitoring a combination of electrocardiogram (“ECG”) signals, blood pressure signals, optical signals, oxygenation signals, oxygen saturation signals, respiration signals, temperature signals, glucose signals, and muscle activity signals.

13. The method of claim 12, wherein the method further comprises the combination to determine the indication.

14. The method of claim 11, wherein the method further comprises monitoring an activity of the animal subject.

15. The method of claim 11, the report indicates a condition of the animal subject.

16. The method of claim 11, wherein the method further comprises monitoring the animal subject continuously or intermittently.

17. A system for monitoring an animal subject, the system comprising:
a. a sensor assembly comprising a plurality of sensors configured for detecting at least physiological signals from a skin of an animal subject;
b. a signal detector configured to generate signal data corresponding to the physiological signals detected by the plurality of sensors; and
c. a controller configured to control the signal detector to monitor the animal subject.

18. The system of claim 17, wherein the sensor assembly further comprises at least one electrode comprising a main body configured to absorb a conductive liquid providing an electrical pathway between a conducting pin on the at least one electrode and the skin of the animal subject.

19. The system of claim 17 wherein the sensor assembly further comprises at least one optical sensor configured to send and receive optical signals adapted to the skin of the animal subject.

20. The system of claim 17, wherein the signal detector is further configured to enhance physiological signals from the skin of the animal subject.
